(19) United States
(12) Patent Application Publication
(10) Pub. No.: US 2004/0048870 A1
(43) Pub. Date: Mar. 11, 2004

(54) Composition for the prevention and treatment of migraine headaches

(76) Inventors: Ahmad A. Amir, San Luis Obispo, CA (US); Mary S. Amir, San Luis Obispo, CA (US)

Correspondence Address: Perkins Coie LLP, P.O. Box 1208, Seattle, WA, 98111-1208, US

(21) Appl. No.: 10/236,381

(22) Filed: Sep. 6, 2002

(51) Int. Cl. A61K 31/525; A61K 31/19; A61K 33/14

(52) U.S. Cl. 514/250; 514/557; 424/682

(57) ABSTRACT

A composition is provided for preventing, treating, and reducing the symptoms of migraine headaches. In one embodiment, the composition includes magnesium and riboflavin as active ingredients. In another embodiment, the composition includes magnesium, riboflavin, and calcium as active ingredients. In yet another embodiment, the composition includes magnesium, riboflavin, calcium, and vitamin D as active ingredients.

### FIELD OF THE INVENTION

The field of the present invention relates to prevention and treatment of migraine headaches.

### BACKGROUND OF THE INVENTION

About 11% of the population in North America suffer from migraine headaches. Migraines can occur in all ages and can appear in children as young as four years of age. The basic causes of migraine headaches are not completely known but appear to be a combination of genetic and environmental factors. Some of these environmental factors, such as stress and hormonal fluctuations, lead to changes in blood flow to the brain and head, changes in hormones in the central nervous system, and electrical imbalances in cells.

Many scientists believe that an underlying central nervous system disorder exists in migraine sufferers. When triggered by various stimuli, the central nervous system sets off a chain of neurologic and biochemical events leading to migraine headache. Some researchers concentrate on a feedback mechanism involving the trigeminal nerve, which runs from the brainstem and branches through the face and jaw. An unknown stimulus triggers the fibers of the nerve to release peptides that produce inflammation and cause nearby blood vessels to swell, stimulating nerves to pulse and fire, and creating the pain of a migraine. An important part of this process involves lower levels of serotonin, that in normal levels, prevent nerve fibers from releasing these inflammatory peptides.

On the other hand, using imaging techniques, a number of studies have noted a drop in blood flow and volume in the brains of patients during migraine attacks. Some scientists believe that migraines occur when blood drains away from the blood vessels in the center of the brain and pools in outer blood vessels. Brain scans of migraine patients reveal that during a migraine attack, blood flow increases in the brainstem. In contrast, the scans show no change in blood flow in the major hemispheres of the brain either during or after the headache. One study suggested that a sudden drop in blood pressure may also precipitate some migraine headaches. Conversely, some cases have suggested that migraine causes hypotension.

Research in migraine headaches has also indicated that slightly over half of migraine cases may be inherited. One study reported that migraines are most common in North America compared to the rest of the world. Migraines are slightly less prevalent in South America and Europe and far less common in Asia and Africa. Investigators believe that the differences are due to genetic variations because, in one study within a single American county, Caucasians had a higher risk than either African Americans or Asians. Researchers also believe that they have located the specific gene for the very rare familial hemiplegics migraine, which they hope may lend some clues to the genetic factors involved with standard migraines.

Hormonal fluctuations have also been associated with migraines. About three times as many women as men have migraines. Although the incidence of migraine is similar for boys and girls during childhood, it increases in girls after puberty. Most migraines in women develop during the hormonally active years between adolescence and menopause. About half of women with migraines report headaches associated with their menstrual cycle. True menstrual migraines, however, may be less common. So-called “true menstrual migraines” do not have auras and occur regularly and only between two days before and three days after menses. The first three months of pregnancy may also exacerbate migraines in some women, although one recent study reported that pregnancy had little effect one way or the other on severity in most women with chronic headaches. Women whose migraines are affected by pregnancy or menstruation are also likely to have worse migraines if they take oral contraceptives. Thus, it appears that although the female hormones, progesterone and estrogen, appear to play some role in migraines, it is their fluctuation, rather than their presence, that is associated with migraines.

Other medical conditions have also been associated with migraines. Certain disorders predispose people to migraines. For example, patients with epilepsy are twice as likely to have migraines as the general population. People who are infected with the bacteria, H. pylori, the major cause of peptic ulcers, or people with fibromyalgia, a syndrome characterized by chronic fatigue and specific muscle pain, also seem to face an increased risk for migraines.

Among the wide range of possible migraine triggers are emotional stress, intense physical exertion, weather changes, bright or flickering lights, high altitude, travel motion, and changes in sleep patterns. More than 100 foods have the capacity to trigger migraine headaches. Low blood sugar has also been known to trigger headaches, and fasting can often precipitate migraines. Likewise, chemicals, such as tyranine, phenylethylamine, tannin, sulfites, or monosodium glutarnate, found in some foods may trigger headache in some people. In children with migraines, common triggers are eating ice cream, anxiety, and fear.

Currently, accepted methods for preventing migraine headaches include identification of headache triggers, dietary modification, exercise, and biofeedback training. Prescription drug therapy for prevention of migraine headaches include, non-steroidal anti-inflammatory drugs (NSAIDs,) beta-blockers, Valproate and other anticonvulsants, antidepressants, calcium-channel blockers, and ergots. For abortive treatment of migraines, medications that are currently available include: over-the-counter headache medications, oral and injectable non-steroidal anti-inflammatory medications (NSAIDS), ergots, and tryptans. These medications, however, may cause significant side effects. In addition, because of the multi-facet etiology of migraines, some medications may work well for some but not for others.

Formulating an effective treatment for any disease, especially one as challenging as migraine headaches, is thus difficult. Some of the difficulties include determining the cause or causes that need to be addressed and how to best address them Although many compounds have been tested against migraine headaches, some with encouraging results, which of these compounds should be used, especially in combination with which others, is most challenging.

### BRIEF SUMMARY OF THE INVENTION

The present invention provides a composition and a method for treating, preventing, and reducing the symptoms of migraine headaches. The composition may include riboflavin and magnesium as the active ingredients. As an alternative, the composition may include riboflavin, magnesium, and calcium as the active ingredients. Yet as another alternative, this composition may include riboflavin, magnesium, calcium and vitamin D as the active ingredients.

All elements of the composition are preferably provided in a single tablet or caplet form suitable for oral ingestion, but may be provided in any other form suitable for administration to humans. Preferably, the composition is taken periodically each day of a treatment period.

### DETAILED DESCRIPTION OF THE PREFERRED EMBODIMENT

The present invention is based on a particular combination of active ingredients, each in a particular dosage range. These active ingredients include magnesium, riboflavin, calcium, vitamin D, and any combination thereof. Tests have shown that the combination of (1) magnesium and riboflavin; (2) magnesium, riboflavin, and calcium; or (3) magnesium, riboflavin, calcium and Vitamin D, have been uniquely effective as a dietary supplement in preventing and treating migraine headaches. In one embodiment, the composition for preventing, treating, or reducing the symptoms of migraine headaches is provided in tablet or caplet form, but can also be provided in other forms including soft-gel capsule, powder or other methods of packaging. Actual formulation into the tablet or caplet form is handled using industry standard methods of production. Preferably, the composition is taken periodically each day of a treatment period or continuously as a dietary supplement.

The details of the individual active components are described below.

Magnesium

Low magnesium levels have been demonstrated in the red blood cells and saliva of migraine sufferers. Reduced intracellular concentrations of magnesium have also been shown in the cortex of migraine subjects using ³¹P-magnetic resonance spectroscopy (P-MRS). Both during and in between attacks, cerebrospinal fluid levels of magnesium are significantly lower in migraine patients compared to normal patients. Thus, magnesium may be involved in the occurrence of migraines. See generally, Gallai, “Serum and salivary magnesium levels in migraine. Results in a group of juvenile patients,” Headache (1992) 32:132-135; Gallai, “Red blood cell magnesium level in migraine patients,” Cephalgia (1993) 13:94-8; Weaver, “Magnesium and migraine,” Headache (1990) 30:168.

In vitro and human studies have shown that low magnesium have several effects that may lead to the occurrence of migraine. Low magnesium level induces cerebral arterial vasospasm and potentiates the contractile response of blood vessels to vasoactive substances such as serotonin. It also enhances the sensitivity of NMDA receptors to glutamate, thus inducing epileptiform discharges and cortical spreading depression. Pro-inflammatory effects have also been observed with increases in platelet aggression leading to serotonin release. Hence, supplementation with magnesium may provide some relief for migraine sufferers.

Administration of magnesium, orally or intravenously, can significantly reduce the frequency of migraine headaches. For example, in a prospective multi-center, placebo controlled double-blind study, Peikert et al. reported that patients orally administered with 600 mg of magnesium versus placebo experienced significant reduction in the frequency of attacks compared to the placebo group. The duration and intensity of the attacks were also decreased in the magnesium group, but not significantly. Peikert, A., et al., “Prophylaxis of migraine with oral magnesium: results from a prospective, multi-center, placebo-controlled and double-blind randomized study,” Cephalgia (1996) 16:257-63. Similarly, in a prospective study of 40 patients, Mauskop et al. showed that intravenous infusion of one gram of magnesium relieved headaches in patients with low serum levels of ionized magnesium. Mauskop et al., “Intravenous Magnesium Sulfate Rapidly Alleviates Headaches of Various Types,” Headache (1996) 36:154-160. Thus, administration of magnesium to migraine sufferers helps reduce the frequency and, in certain situations, the duration and intensity of migraine attacks.

Riboflavin (Vitamin B₂)

Migraine pathogenesis may also involve mitochondrial dysfunction that results in impaired oxygen metabolism. The brains of migraine headache sufferers are characterized between attacks by a reduction of mitochondrial phosphorylation potential. Riboflavin, which has the potential of increasing mitochondrial energy efficiency, has a prophylactic effect in treating migraine headaches.

For example, Schoenen et al. showed the effects of administering high-dose riboflavin to migraine suffering patients in a double-blind randomized multi-center trial involving 55 patients (28 in study and 27 placebo). The study patients were placed on 400 mg of oral riboflavin taken as caplets once a day. In the study group, there was a statistically significant decrease, at four months, in the number of days the patients experienced migraine attacks. Schoenen, J., et al., “Effectiveness of high-dose riboflavin in migraine prophylaxis: A randomized controlled trial,” Neurology (1998) 50:466-470. Thus, riboflavin can be administered to migraine sufferers to alleviate the frequency of the headaches.

Calcium and Vitamin D

Some studies have suggested that migraine is a cyclic vasomotor phenomenon. Alterations in cellular calcium homeostasis may have an important role in vasospasm and ischemic injury in the brain, heart, and kidney. Thus, by regulating smooth muscle contraction and mediating the excitability of nervous tissues, calcium may be able to alleviate migraines.

For example, Thys-Jacobs reported that on two post-menopausal women, calcium and vitamin D supplementation reduced the frequency and duration of their migraine headaches. Thys-Jacobs, S., “Vitamin D and calcium in menstrual migraine,” Headache (1994) 34:544-546; Thys-Jacobs, S., “Alleviation of migraine with therapeutic vitamin D and calcium,” Headache (1994) 34:590-592. Thus, calcium and vitamin D may be administered to migraine sufferers to alleviate their headaches.

Formulation and Dosage

Although these previous studies show that magnesium, riboflavin, calcium, and vitamin D may separately help alleviate migraine, none of these studies ever combined these chemicals together to form a single formulation. In one embodiment of the present invention, magnesium and riboflavin are formulated together in a single tablet or caplet suitable for oral administration to migraine sufferers. Preferably, the composition is taken periodically (e.g., once or twice) each day of a treatment period, [which is usually 60 days.] Alternatively, the composition can be a dietary supplement that is taken continually and indefinitely.

The magnesium component may be in the form of a magnesium salt. For example, suitable sources of magnesium salts include, but are not limited to magnesium chloride, magnesium citrate, magnesium gluceptate, magnesium gluconate, magnesium hydroxide, magnesium lactate, magnesium oxide, magnesium sulfate, magnesium lactate, magnesium pidolate, magnesium carbonate, and magnesium tartarate and mixtures thereof. The magnesium/riboflavin composition in this embodiment may include one or more of these magnesium salts.

The dosage of magnesium in the composition may be in the range of 100-800 mg of elemental magnesium, preferably in the 200-600 mg range. Elemental magnesium means that the mass of the dosage (e.g., 400 mg) refers only to the magnesium component of an ionic compound such as magnesium chloride. In the most preferred embodiment, magnesium is provided in 200 mg per tablet. Although oral magnesium may cause diarrhea, which usually resolves with time, and is contraindicated in individuals with impaired kidney function, magnesium is a very safe dietary supplement.

The dosage of riboflavin in the composition may be in the range of 100-600 mg, Preferably between 300-500 mg, and most preferred at a dose of 400 mg per tablet. Although riboflavin may cause urine to have a more yellow color than normal, especially if large doses are taken, the safety profile of riboflavin is excellent and usually does not cause any side effects.

In another embodiment, calcium may also be included in the formulation together with magnesium and riboflavin. Suitable sources of calcium for the composition include, but are not limited to: calcium carbonate, calcium citrate, calcium glubionate, calcium gluceptate, calcium gluconate, calcium lactate, calcium lactate-gluconate, calcium phosphate, dibasic, calcium phosphate, tribasic, or any mixtures thereof. The dosage of calcium in the composition may be in the range of 500-2,000 mg elemental calcium, preferably between 1,000-1,500 mg. Most preferably, the composition includes 1,200 mg of elemental calcium, which can be derived from one or more of the calcium salts. The frequency of administration may be similar to that of the magnesium and riboflavin alone.

In another embodiment, vitamin D may also be included in the composition together with magnesium, riboflavin, and calcium. This may be especially useful in conditions when calcium metabolism may be affected by lack of vitamin D. Suitable forms of vitamin D include alfacalcidol, calcifediol, calcitriol, dihydrotachysterol, or ergocalciferol.

For alfacalcidol, the dosage in the composition may be in the range of 1 to 5 micrograms, preferably one microgram. For calcifediol, the dosage in the composition may be in the range of 40 to 100 micrograms, preferably 50 micrograms. For calcitriol, the dosage in the composition may be in the range of 0.25 to 0.5 micrograms, preferably 0.25 micrograms. For-dihydrotachysterol, the dosage in the composition may be in the range of 100 micrograms to 300 milligrams, preferably one milligram. For ergocalciferol, the dosage in the composition may be in the range of 100 to 500 units, preferably 200 units.

In all of the above embodiments, inactive elements, which are well known in the art, are preferably provided as fillers to put the active elements in tablet or caplet form. For example, the fillers may include binders, lubricants, and disintegrants, which could include cellulose, gelatin, and silica.

### EXAMPLE 1

The patient is a 56-year-old female with a history of migraine headaches since puberty. Prior to institution of therapy, a headache questionnaire administered to the patient yielded the following results: Headaches were characterized by throbbing pain, primarily on the left, accompanied by photophobia, phonophobia, nausea and occasional vomiting. Triggers included xanthines (caffeine and chocolate), sleep deprivation, and emotional stress. The headaches had a catamenial component in that they are worse several days prior to menses. Intensity, on a scale of 0-10 with 10 being the worst pain ever experienced, was rated at 8-9/10 lasting for several days at a time. There were stretches during the month when she had symptoms lasting 3-4 days at a time. She identified on a headache calendar that she had on average six or more headaches rated between 5-10 per month, and a total 12 headaches per month. She had not only missed work on occasion but also had visited the emergency department due to the intractable nature of her symptoms. She had tried multiple over-the-counter abortive agents for acute pain including acetaminophen, aspirin, ibuprofen, all of which provided some, but incomplete, relief.

The patient was started on a combination of 200 mg of oral magnesium, 400 mg oral riboflavin and 1200 mg oral calcium, taken once daily. After an initial 60 day treatment period, a headache questionnaire administered to the patient showed the following: The frequency of the headaches has decreased to 1-2 times per month. Duration of symptoms per attack has been decreased to 4-6 hours. The severity of headaches are now rated as 2-3 (scale of 1-10, 10 being the worst headache she has had). She has not missed any days at work due to headaches following the initial treatment period which are now easy to abort with the aforementioned common over-the-counter medications. This patient has continued on a maintenance regimen of 200 mg of oral magnesium, 400 mg oral riboflavin and 1200 mg oral calcium, taken as a daily dietary supplement.

### EXAMPLE 2

The patient is a 38-year-old male with a history of migraine headaches since the age of 25. Prior to institution of therapy, a headache questionnaire administered to the patient yielded the following results: Headaches were described as occurring almost daily for periods of 3-4 weeks at a time with several weeks in between as pain-free. The headaches were characterized as being heralded by visual scotoma, “sparkles” making it difficult to read for a 30-minute period, followed by intense pulsating pain. There were no accompanying autonomic symptoms. Intensity, on a scale of 0-10 with 10 being the worst pain ever experienced, was rated at 6-7/10, with a duration of at least 6 hours but frequently it would last days. Triggers included red wine, nitrites, emotional stress and sleep deprivation. The patient was not exposed to xanthines. The patient had taken beta blockers, inhalers, Midrin, Imitrex inhalers and injections, Fiorinal and Demerol injections, all of which had varying success with varying side effects.

The patient was started on a once-daily regimen of 200 mg of oral magnesium and 400 mg of oral riboflavin. After an initial treatment period of 60 days, a follow-up headache questionnaire was administered to the patient. The headache profile improved with a decreased intensity going from 6-7/10 to 1-3/10 when they did occur, which was less than one per week and were much more amenable to abortive treatment than they had been previously The duration of the headaches also improved going from >6 hours to days, to <4 hours. He has continued on a regimen of 200 mg of oral magnesium and 400 mg of oral riboflavin, taken as a daily dietary supplement.

From the foregoing, it will be appreciated that specific embodiments of the invention have been described herein for purposes of illustration, but that various modifications may be made without deviating from the spirit and scope of the invention. Accordingly, the invention is not limited except as by the appended claims. The preceding Examples are intended only as examples and are not intended to limit the invention. It is understood that modifying the examples above does not depart from the spirit of the invention. It is further understood that each example may be applied on its own or in combination with other examples.

1. A composition for preventing and treating migraine headaches in a human, the composition consisting essentially of magnesium and riboflavin.

2. The composition in claim 1 wherein the magnesium is formulated at a dosage ranging from 100 to 800 mg elemental magnesium.

3. The composition in claim 2 wherein the magnesium is formulated at a dosage ranging from 200 to 600 mg elemental magnesium.

4. The composition in claim 3 wherein the magnesium is formulated at a dosage of 200 mg elemental magnesium.

5. The composition in claim 1 wherein the magnesium is selected from magnesium salts of the group consisting of magnesium chloride, magnesium citrate, magnesium gluceptate, magnesium gluconate, magnesium hydroxide, magnesium lactate, magnesium oxide, magnesium sulfate, magnesium lactate, magnesium pidolate, magnesium carbonate, and magnesium tartarate.

6. The composition of claim 1 wherein the riboflavin is formulated at a dosage ranging from 100 to 600 mg.

7. The composition of claim 6 wherein the riboflavin is formulated at a dosage of 400 mg.

8. The composition of claim 1 wherein the magnesium is formulated at 200 mg elemental magnesium and riboflavin is formulated at 400 mg.

9. The composition of claim 1 wherein the composition is formulated for oral administration.

10. The composition in claim 9 wherein the composition is provided in a single from selected from a group consisting of caplet, tablet, soft-gel capsule and powder capsule.

11. The composition of claim 1 wherein the composition is administered on a periodic basis each day of a treatment period.

12. The composition of claim 11 wherein the period of administration in each day is one or two times.

13. A composition for preventing and treating migraine headaches in human, the composition comprising magnesium, riboflavin, and calcium.

14. The composition of claim 13 wherein the calcium is formulated at a dosage ranging from 500-2000 mg elemental calcium.

15. The composition of claim 14 wherein the calcium is formulated at 1,200 mg elemental calcium.

16. The composition of claim 14 wherein calcium is selected from calcium salts of the group consisting of calcium carbonate, calcium citrate, calcium glubionate, calcium gluceptate, calcium gluconate, calcium lactate, calcium lactategluconate, calcium phosphate, dibasic, and calcium phosphate, tribasic.

17. The composition of claim 13 wherein the magnesium is formulated at 200 mg elemental magnesium, riboflavin is formulated at 400 mg, and calcium is formulated at 1,200 mg elemental calcium.

18. The composition in claim 13 wherein the magnesium is formulated at a dosage ranging from 100 to 800 mg elemental magnesium.

19. The composition in claim 18 wherein the magnesium is formulated at a dosage ranging from 200 to 600 mg elemental magnesium.

20. The composition in claim 19 wherein the magnesium is formulated at a dosage of 200 mg elemental magnesium.

21. The composition in claim 20 wherein the magnesium is selected from a group consisting of magnesium chloride, magnesium citrate, magnesium gluceptate, magnesium gluconate, magnesium hydroxide, magnesium lactate, magnesium oxide, magnesium sulfate, magnesium lactate, magnesium pidolate, magnesium carbonate, and magnesium tartarate.

22. The composition of claim 13 wherein the riboflavin is formulated at a dosage in ranging from 100 to 600 mg.

23. The composition of claim 22 wherein the riboflavin is formulated at 400 mg.

24. The composition of claim 13 wherein the composition are formulated for oral administration.

25. The composition in claim 24 wherein the composition is provided in a single form selected from a group consisting of caplet, tablet, soft-gel capsule and powder capsule.

26. The composition of claim 13 wherein the composition is administered on a periodic basis each day of a treatment period.

27. The composition of claim 26 wherein the period of administration in each day is one or two times.

28. A composition for preventing and treating migraine headaches in human, the composition comprising magnesium, riboflavin, calcium, and vitamin D.

29. The composition in claim 28 wherein the vitamin D is in a form selected from the group consisting of alfacalcidol, calcifediol, calcitriol, dihydrotachysterol, and ergocalciferol.

30. The composition in claim 29 wherein the dosage of alfacalcidol in the composition is in the range of 1 to 5 micrograms.

31. The composition in claim 29 wherein the dosage for calcifediol in the composition is in the range of 40 to 100 micrograms.

32. The composition in claim 29 wherein the dosage for calcitriol in the composition is in the range of 0.25 to 0.5 micrograms.

33. The composition in claim 29 wherein the dosage for dihydrotachysterol in the composition is in the range of 100 micrograms to 3.0 milligrams.

34. The composition in claim 29 wherein the dosage for ergocalciferol in the composition is in the range of 100 to 500 units.

35. The composition of claim 28 wherein the composition are formulated for oral administration.

36. The composition in claim 35 wherein the composition is provided in a single form selected from a group consisting of caplet, tablet, soft-gel capsule, and powder capsule.

37. The composition of claim 28 wherein the composition is administered on a periodic basis each day of a treatment period.

38. The composition of claim 37 wherein the period of administration in each day is one or two times.
